(19) United States
(12) Patent Application Publication
(10) Pub. No.: US 2005/0038473 A1
(43) Pub. Date: Feb. 17, 2005

(54) Device and method for the treatment of pilosebaceous disorders

(76) Inventors: Dov Tamarkin, Maccabim (IL); Dnnicln Mavor, Tel Aviv (IL); Noum Emanuel, Jerusalem (IL)

Correspondence Address: KENYON & KENYON, 1500 K STREET, N.W., SUITE 700, WASHINGTON, DC, 20005, US

(21) Appl. No.: 10/890,235

(22) Filed: Jul. 14, 2004

Related U.S. Application Data
(60) Provisional application No. 60/487,303, filed on Jul. 14, 2003.

(51) Int. Cl. A61N 1/30

(52) U.S. Cl. 607/3

(57) ABSTRACT

The present invention discloses a device, kit and a method for the treatment of a disorder of the pilosebaceous unit. Moreover, a device and kit for treatment of a hair growth disorder is disclosed. The kit for treating a hair growth disorder may comprise at least one formulation comprising a hair growth modulating agent and at least one electrically powered device for promoting delivery of the hair growth modulating agent into and/or onto the body area. Preferably, the device is a thin and flexible patch. Methods of using such a device/kit for treatment of a disorder of the pilosebaceous unit is also disclosed.

### CROSS REFERENCE TO RELATED APPLICATION

This application claims priority to U.S. Provisional Application No. 60/487,303, filed Jul. 14, 2003, the content of which is incorporated herein by reference in its entirety.

### FIELD OF THE INVENTION

The present invention relates to a kit, device and a method for the treatment of a hair growth disorder.

### BACKGROUND OF THE INVENTION

Hair grows in cycles, which are not synchronized in human beings; each hair enters phases of the growth cycle at a different time. There are three phases of the hair growth cycle: anagen, catagen and telogen. Anagen is the phase of active hair growth—approximately 90% of all hairs are in anagen. It lasts from 2 to 6 years, depending on skin region. After anagen is completed, the hair enters catagen; during this short phase (2-3 weeks) the matrix cells gradually stop dividing and eventually keratinize. When full keratinization is achieved, the hair enters the last phase of the cycle, telogen. During the telogen phase (3-4 months) keratinized hair falls out, and a new matrix is gradually formed from stem cells in the basal layer of the outer epithelial root sheath bulge. A new hair starts to grow and the follicle is back in anagen phase.

Stem cells of the hair follicle are gathered in the basal layer of the outer root sheath bulge. It is from these cells that matrix cells are formed. Growth and differentiation of the matrix cells are under the influence of substances produced by cells of the dermal papilla. On the other hand, the secretory activity of the dermal papilla is controlled either by substances produced in cells of the spinous layer of the outer root sheath or by hormones. Cells of the spinous layer produce peptides greater than 3000 daltons, which increase the number of papilla cell mitoses two to five times. It was recently discovered that basic fibroblast growth factor (bFGF) and platelet-derived growth factor (PDGF) potentiate the growth of dermal papilla cells. It is proposed that these proteins increase the synthesis of stromelysin (an enzyme, matrix metalloproteinase), which acts on the papilla cells and accelerates their growth.

Another cytokine, transforming growth factor beta, inhibits mitogen-induced dermal papilla cell proliferation. On the other hand, dermal papilla cells produce numerous cytokines, which influence proliferation of hair matrix cells. Some of these are stimulators, and some inhibitors. Interleukin 1-alpha (IL-1-alpha) inhibits growth of hair and follicle, but only after a latency period of 2-4 days. The increase of IL-1-alpha concentration in extracellular fluid during inflammation could be one of the reasons for alopecia following certain infectious diseases. Apart from IL-1-alpha, both fibroblast growth factor (FGF) and epidermal growth factor (EGF) inhibit growth of the hair and hair follicle. Fibroblast growth factor type 5 (FGF5) is an especially potent inhibitor. Receptors for these ligands have been found by immunohistochemical methods on papilla cells, matrix cells and stem sells in the bulge region of the hair follicle. An additional cytokine produced by cells of the dermal papilla, keratinocyte growth factor (KGF), induces extensive hair growth in murine models of alopecia. Receptors for KGF were found on keratinocytes in the basal epidermis and throughout developing hair follicles of rat embryos and neonates.

Insulin-like growth factor I (IGF-I) accelerates, in a concentration-dependent manner, growth of hair and hair follicles. The actions of IGF-I are modulated by proteins produced in dermal papilla cells, which bind IGF (insulin-like growth factor-binding proteins: IGFBPs); the exact mechanism of modulation has not yet been resolved. However, it has been shown that IGFBP-3 (which is the most abundant IGFBP type in dermal papilla cells) forms a complex with free IGF-I to reduce the concentration of IGF-I available for stimulation of hair elongation and maintenance of the anagen phase. Retinoids and glucocorticoids stimulate production of IGFBP-3 in dermal papilla cells. Insulin itself has the same effect as IGF-I; it has been observed that body hair in patients with hyperinsulinism, has a male distribution pattern. On the other hand, growth hormone (somatotropin) has no direct influence on follicle and hair growth.

Animal studies have shown that substance P induces transition of hair from telogen to anagen phase. The same effect has been observed with the active principle of chili peppers, capsaicin, which releases substance P from nerve endings in skin.

Substances regulating the homeostasis of calcium and phosphorous may also be involved in control of hair growth. Parathyroid hormone (PTH) and PTH-related peptide inhibit hair growth and epidermal cell proliferation. 1,25-dihydroxyvitamin D3 (1,25/OH/D3) in low concentration (1-10 nM) stimulates, and in high concentration (100 nM) and after longer contact inhibits hair and hair follicle growth. These actions of PTH and 1,25/OH/D3 require direct contact with hair follicles.

Androgens have diverse effects on hair in different body regions. Effects vary from essentially nonexistent (e.g. on eye-lashes), weak (on temporal and suboccipital region hair), moderate (on extremity hair), or strong (on facial, parietal region, pubic, chest, and axillary hair) effects. Androgens bind to receptors both in the cytoplasm and nuclei of dermal papilla cells and some cells of the sheaths of the follicle, but only if the hair is in anagen or telogen. Two molecular forms of androgen receptors have been proposed: active (protein-monomer, 62 kDa) and inactive (protein-tetramer, with four subunits, total molecular weight 252 kDa). The monomer form has much greater affinity for androgens (dissociation constant for dihydrotestosterone is 2.9 nM). Four monomer molecules aggregate to form a tetramer in a reversible reaction. Necessary factors are glutathione and the enzyme, endogenous disulfide converting factor. The complex of androgen hormone-receptor moves to the cell nucleus and there enables expression of genes coding cytokines. Cells of the dermal papilla synthesize and secrete cytokines, which control growth and differentiation of hair matrix cells. In most hair the released cytokines stimulate matrix cell division and differentiation, however for hair of the parietal region the cytokines act as inhibitors, leading to follicle atrophy.

Numerous factors affect the number and activity of androgen receptors in dermal papilla cells. Retinoic acid (vitamin A derivative), if used for a long time, may reduce the number of androgen receptors by 30-40 percent. Vitamin B6 reduces by 35-40% the extent of protein synthesis observed after androgen receptor activation. A polypeptide with molecular weight of 60 kDa, analogous to an intracellular calcium-binding protein called calreticulin, prevents binding of the androgen-receptor complex to DNA and also results in the production of calreticulin.

Among all androgens, dermal papilla cells are most affected by 5-alpha-dihydrotestosterone (5-alpha-DHT). It is synthesized in these cells from testosterone under catalytic action of the enzyme 5-alpha-reductase.

Growth of androgen-dependent hair can be influenced in several ways: (a) by decreasing androgen production, (b) by blocking testosterone transformation to 5--DHT or (c) by blocking androgen receptors. Androgen production can be decreased either surgically (removal of hormone-producing ovarian or adrenal tumor) or with drugs. If increased production of androgens is the consequence of adrenal cortex hyperplasia, it can be suppressed with cortisone. Exogenous cortisone will inhibit release of ACTH from the hypophysis, and this in turn will decrease hyperplasia. If increased androgen production is caused by polycystic ovarian dystrophy, it can be reduced by inhibition of hypophyseal release of gonadotropins. Continuous administration of gonadorelin analogs (leuprolide, goserelin, decapeptyl, etc.) is a very efficient tool for achieving this goal. However, administration of these drugs is accompanied by significant adverse effects that result from decreased estrogen and progesterone production. Menstrual irregularities, flushes and osteoporosis are commonly observed.

Transformation of testosterone to 5-alpha-DHT can successfully be interrupted with inhibitors of 5-alpha-reductase. One of them, finasteride, is already used clinically with significant efficacy and without disturbance of sex hormone plasma levels. Finasteride only inhibits type II 5-alpha-reductase. There are other 5--reductase blockers (so-called azasteroids), that have a steroid nucleus with an attached 4-methyl-4-azo moiety and a long hydrophobic side chain on C-17. The most efficient among them is 17-beta-N,N-diethylcarbamyl-4-methyl-4-aza-5-androstan-3-one, which has a greater effect on in-vitro hair follicle cultures than finasteride.

One way to suppress the growth of androgen-dependent hairs is by the blockade of androgen receptors. The competitive androgen receptor blocker flutamide has already been approved for human use. Women with idiopathic hirsutism taking flutamide experienced a 30% reduction of hair diameter without disturbance of plasma levels of gonadotropins, testosterone, androstenedione or dehydroepiandrostenedione.

Several blockers of androgen receptors with non-steroid chemical structure were synthesized recently. They are N-substituted arylthiohydantoins: RU 59063, RU 56187 and RU 58841. These are very potent substances. Their affinity for androgen receptors is three times higher than the affinity of testosterone. One of them, RU 58841, is active when applied locally, which is of great benefit considering the significant adverse effects observed after systemic administration. One of the imidazole antimycotics, ketoconazole, is an inhibitor of androgen biosynthesis and also an androgen receptor blocker, however its affinity for androgen receptors is low. Systemic administration of ketoconazole for the treatment of hirsutism requires high doses and is associated with a high incidence of adverse effects.

Despite intensive research and development efforts, as outlined in the above introduction and in the background art, there is no available satisfactory treatment for hair growth disorders. The lack of effective treatment is, at least partially, attributed to insufficient dermal penetration of active agents into the pilosebaceous unit (encompassing the sebaceous gland and hair follicle), which is the target site of action for such active agents.

Iontophoresis has been known for many years, as a means to deliver drugs and cosmetic active agents into the skin for therapeutic purposes. It is based on mechanisms, which include (a) iontophoresis, in which a charged ion is repelled from an electrode of the same charge, and (b) electroosmosis, based on the convective movement of solvent that occurs through a charged “pore” in response to the preferential passage of counter-ions when the electric field is applied. In the context of the present invention, the term “iontophoresis” will stand for any method of electrical dermal delivery of substances, including iontophoresis, electroosmosis electrotransport, and electroporation and any combination thereof.

Devices that deliver active substances using iontophoresis have been developed for many applications, most of which involve the delivery of pharmaceutical compounds through the subject's skin and into the circulatory system or other organs of a subject's body. Topical application of one or more active ingredient to the skin through the use of an iontophoresis device is called dermal treatment.

It is therefore desirable to have the benefit of a device, kit and method of use thereof for the treatment of a hair growth disorder that would be effective, upon topical administration. Enhanced dermal penetration is highly desirable in this respect. It is further desirable to have such a device, kit and method of use thereof, which does not involve oral administration of an active agent. Furthermore, it is desirable to allow the subject, with a hair growth disorder to perform such treatment individually, in the home setting, without the need for professional help. Finally, it is desirable to have such a system, which has low cost. Preferably, such a device should be disposable.

### SUMMARY OF THE INVENTION

Embodiments of the present invention include kits, devices and methods for treatment of a disorder of the pilosebaceous unit. Embodiments of the kits may comprise an electrically powered patch and a formulation comprising a hair growth modulating agent. Embodiments of the devices of the present invention may comprise first and second electrodes, a power source and a hair growth modulating agent. Preferably the device is a thin and flexible patch. Embodiments of the methods of the present invention may comprise providing a device for treatment of a hair growth disorder, contacting the body area with the device for a time period wherein the device promotes hair growth modulating agent penetration of the body area surface and underlying tissues, to treat the hair growth disorder, and removing the device from the body area. Additional embodiments of the methods of the present invention may also comprise providing a device for treatment of a hair growth disorder, contacting the body area with the device for a time period wherein the device provides electrical stimulation to the body area, electrically stimulating the body area to treat the hair growth disorder, and removing the device from the body area.

In a first embodiment the present invention provides a kit for treating a hair growth disorder, the kit comprising: (a) at least one formulation comprising a hair growth modulating agent; and (b) at least one electrically powered device for promoting delivery of the hair growth modulating agent into and/or onto the body area.

In a second embodiment the present invention provides a kit for treating a disorder of the pilosebaceous unit, comprising: (a) at least one formulation comprising an androgen inhibitor; and (b) at least one electrically powered device for promoting delivery of the androgen inhibitor into and/or onto a body area comprising pilosebaceous units.

In a third embodiment the present invention provides a device for treatment of a disorder of the pilosebaceous unit comprising a flexible, wearable patch conformable to the contour of a body area surface and comprising: (a) at least one first electrode adapted to communicate an active substance into the body area by application of an electrical current on the body area surface; (b) at least one second electrode, facilitating closing of electrical circuit with the body area surface; (c) at least one power source for providing a current and voltage, connected through a conductive media to the first and second electrode; and (d) a hair growth modulating agent and or an anti-acne agent for treatment of the disorder.

In a preferred embodiment the device is an iontophoretic patch.

In a preferred embodiment the patch comprises: (a) at least one first electrode in electrical contact with a first region of the body area, (b) at least one second electrode in electrical contact with a second region of the body area; and (c) at least one power supply for supplying electrical energy to the first electrode and the second electrode, wherein the power supply is supported on a base member.

In a preferred embodiment the power supply comprises at least one electrochemical cell.

In a preferred embodiment the electrochemical cell is thin and flexible.

In a preferred embodiment the electrochemical cell is up to 1 mm thick.

In a preferred embodiment the first electrode is an anode and the second electrode is a cathode.

In a preferred embodiment the electrochemical cell comprises: (a) a first layer of insoluble negative pole; (b) a second layer of insoluble positive pole; and (c) a third layer of aqueous electrolyte disposed between the first and second layers and including (i) a deliquescent material for keeping the electrochemical cell wet at all times; (ii) an electroactive soluble material for obtaining ionic conductivity; and (iii) a water-soluble polymer for obtaining a desired viscosity for adhering the first and second layers to the third layer.

In a preferred embodiment the electrochemical cell has open architecture.

In a preferred embodiment the patch comprises an active electrode, and the active electrode is the anode, the cathode or both the anode and the cathode.

In a preferred embodiment the formulation is disposed on the active electrode.

In a preferred embodiment the device further comprises a holding component for holding and storing the formulation and having a pore size and a pore density, the holding component contacting a first region of the body area and the active electrode.

In a preferred embodiment the holding component is selected from the group consisting of a sponge, separator, non-woven material or substrate base layer.

In a preferred embodiment the hair growth modulating agent is applied directly to the body area.

In a preferred embodiment the hair growth modulating agent is disposed in the interface area between the electrodes.

In a preferred embodiment the kit promotes surface treatment, dermal treatment and transdermal treatment of the body area with the hair growth modulating agent.

In a preferred embodiment the kit further comprises a conductive layer comprising a conductive composition disposed between the patch and the body area, for providing a conductive interfacing layer between the patch and the body area.

In a preferred embodiment the conductive composition is a conductive adhesive.

In a preferred embodiment the hair growth modulating agent is contained in the conductive composition.

In a preferred embodiment the patch comprises an active electrode and a counter electrode, and the conductive layer is disposed on the active electrode.

In a preferred embodiment the treatment is selected from the group consisting of curing, alleviating condition, alleviating symptoms, prevention and a combination thereof.

In a preferred embodiment the patch is thin and flexible.

In a preferred embodiment the patch is made by a printing method.

In a preferred embodiment the patch further comprises attachment means.

In a preferred embodiment the hair growth disorder is selected from the group consisting of any type of hair loss and excessive hair growth, acne, male pattern baldness, androgenic alpecia, alopecia greata, alopecia totalis and alopecia universalis and a combination thereof.

In a preferred embodiment the hair growth modulating agent is selected from the group consisting of a hair stimulating agent, a hair growth inhibitory agent, a hair removal agent and a combination thereof.

In a preferred embodiment the hair growth modulating agent is selected from the group consisting of androgen inhibitors, inhibitors of omithine decarboxylase, S-adenosylmethionine decarboxylase, gamma-glutamyl transpeptidase, and transglutaminase, keratin inhibitors, hair growth stimulants, vitamins, growth factors, cytokines, potassium formulations, corticosteroids, minerals, immunomodulators and any combination thereof.

In a preferred embodiment the hair growth modulating agent is an agent, which acts directly on the hair follicle.

In a fourth embodiment the present invention provides a device for treatment of a disorder of the pilosebaceous unit comprising a flexible, wearable patch conformable to the contour of a body area surface and comprising: (a) at least one first electrode adapted to communicate an active substance into the body area by application of an electrical current on the body area surface; (b) at least one second electrode, facilitating closing of electrical circuit with the body area surface; and (c) at least one power source for providing a current and voltage, connected through a conductive media to the first and second electrode; wherein the electrical current stimulates hair growth. In a preferred embodiment the formulation comprises potassium. In a preferred embodiment the device further comprises a substrate base layer.

In a fifth embodiment the present invention provides a method for treating a hair disorder of a body area with an agent for modulating hair growth comprising the steps of: providing at least one electrically powered device for promoting delivery of the hair growth modulating agent into and/or onto the body area: contacting the body area with the device for a time period wherein the device promotes hair growth modulating agent penetration of the body area surface and underlying tissues; penetrating of the hair growth modulating agent into and/or onto the body area to treat the hair disorder; and removing the device from the body area. In a preferred embodiment the formulation comprising the hair growth modulating agent is applied onto affected body area prior to the step of contacting the body area with the device. In a preferred embodiment the formulation comprising the hair growth modulating agent is applied onto affected body area simultaneously with the device. In a preferred embodiment the formulation is disposed on the device.

In a sixth embodiment the present invention provides a method for treating a disorder of the pilosebaceous unit comprising the steps of: (a) providing at least one electrically powered device; (b) contacting the pilosebaceous unit body area with the device for a time period wherein the device provides electrical stimulation to the body area; (c) stimulating the pilosebaceous unit body area to treat the disorder; and (d) removing the device from the body area. In a preferred embodiment the method further comprises the step of applying a formulation comprising potassium to modulate hair growth.

In a preferred embodiment the hair growth disorder is baldness. In a preferred embodiment the hair growth disorder is selected from the group consisting of male pattern baldness, androgenic alpecia, alopecia greata, alopecia totalis and alopecia universalis and a combination thereof. In a preferred embodiment the hair growth disorder is excessive hair growth. In a preferred embodiment the hair growth disorder is acne. In a preferred embodiment the device comprises: (a) at least one first electrode in electrical contact with a first region of the body area, (b) at least one second electrode in electrical contact with a second region of the body area; and (c) at least one power supply for supplying electrical energy to the first electrode and the second electrode, wherein the power supply is supported on a base member. In a preferred embodiment the power supply comprises at least one electrochemical cell. In a preferred embodiment the electrochemical cell is thin and flexible. In a preferred embodiment the electrochemical cell comprises: (a) a first layer of insoluble negative pole; (b) a second layer of insoluble positive pole; and (c) a third layer of aqueous electrolyte disposed between the first and second layers and including (i) a deliquescent material for keeping the electrochemical cell wet at all times; (ii) an electroactive soluble material for obtaining ionic conductivity; and (iii) a water-soluble polymer for obtaining a desired viscosity for adhering the first and second layers to the third layer.

The term ‘treatment’ as used herein refers to treating, curing, preventing and alleviating symptoms of a disorder.

The term ‘hair growth modulating agent’ as used herein includes, but is not limited to a compound or substance, which can change the amount or rate of growth of hair, including preventing or retarding hair growth, removing hair, stimulating hair growth, act directly on the hair follicle or a combination thereof.

The term ‘pilosebaceous unit’ as used herein includes sebaceous glands and hair follicles, which are physically connected and usually vary inversely in size. Pilosebaceous units are located in all skin areas except the palms of the hands and soles of the feet.

The term ‘disorders of the pilosebaceous unit’ as used herein includes any type of physical or physiological disorder associated with the sebaceous glands and or hair follicles. The term includes, but is not limited to acne, acne related disorders, hair disorders, hair growth disorders, excessive hair growth, lack of hair growth and combinations thereof.

The term ‘excessive hair growth’ as used herein includes too much hair growth in any body area. The term also includes undesired body hair growth, which is not necessarily the result of a hair growth disorder, such as, but not limited to hair growth under the arm pits, facial hair, hair on the arms or legs and pubic regions.

### BRIEF DESCRIPTION OF DRAWINGS

With reference now to the drawings in detail, it is stressed that the particulars shown, are by way of example and for the purposes of illustrative discussion of the preferred embodiment of the present invention only, and are presented for providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

FIG. 1 is a schematic drawing of an embodiment of an iontophoretic patch according to one embodiment of the present invention.

FIG. 2 is a flow diagram of a method of using a device or kit according to one embodiment of the present invention.

### DETAILED DESCRIPTION OF THE EMBODIMENTS OF THE INVENTION

The present invention is of a kit, device and method for treating a disorder of the pilosebaceous unit, such as but not limited to a hair growth disorder. Preferably, the kit includes an active agent, which is effective in the treatment of a hair growth disorder and an electrically powered device. According to one preferred embodiment of the present invention there is provided a kit, wherein the kit comprises (a) a formulation, containing active agents, effective in the treatment of a hair growth disorder; and (b) an electrically powered device, such as an iontophoretic patch, which comprises an electrochemical cell having at least two electrodes positioned on one side of the dermal device, for forming electrical contact with the skin portion of the subject; the device, being designed and configured for delivering an electric current for introduction of current and/or voltage to the skin portion of the subject and for promoting delivery of the active agent. Any suitable electrically powered device can be used such as, but not limited to a passive or active iontophoresis device, a TENS device, a galvanic stimulation device, an Interferential current device or any suitable combination thereof. Preferably, electrically powered device is a thin and flexible patch device. Most preferably, the electrically powered device is an iontophoresis device. Use of the present invention is intended to provide an effective treatment of a hair growth disorder or other suitable disorder of the pilosebaceous unit.

The advantages of such a device, kit and method of use thereof as provided by the present invention for treatment of a hair growth disorder is multiple fold: The kit can readily facilitate enhancement of the availability of the active agents in the skin, thereby increasing their effectiveness. Furthermore, the kit provides topical administration, with increased efficacy and minimal systemic exposure, thereby enhancing the safety and efficacy at the same time. Still further the kit of the present invention can optionally be a disposable kit, which can be used at home.

Preferably, the electrically powered device is a flexible, wearable electrically powered device, such as an iontophoretic patch, that can conform and adhere to the skin surface of a person. Preferably, the device further includes a first and a second electrode connected to a power source. The device, such as an iontophoretic patch, is adapted to deliver an active agent, suitable for the treatment of a hair growth disorder. The patch may be in any suitable size, shape and design. Preferably, the patch is ergonomic and is configured to fit the contours of a body surface, such as, but not limited to in the form of a strip, patch, mask, cap, (for example shower cap or skullcap design) or combination thereof.

Before explaining at least one embodiment of the invention in detail, it is to be understood that the invention is not limited in this application to the details of construction and the arrangement of the components set forth in the following description or illustrated in the drawings. The invention is applicable to other embodiments or of being practiced or carried out in various ways. Also, it is to be understood that the phraseology and terminology employed herein is for the purpose of description and should not be regarded as limiting.

The principles and operation of kits, devices and methods of use thereof according to the present invention may be better understood with reference to the figures. The figures show two embodiments of the present invention and are not limiting.

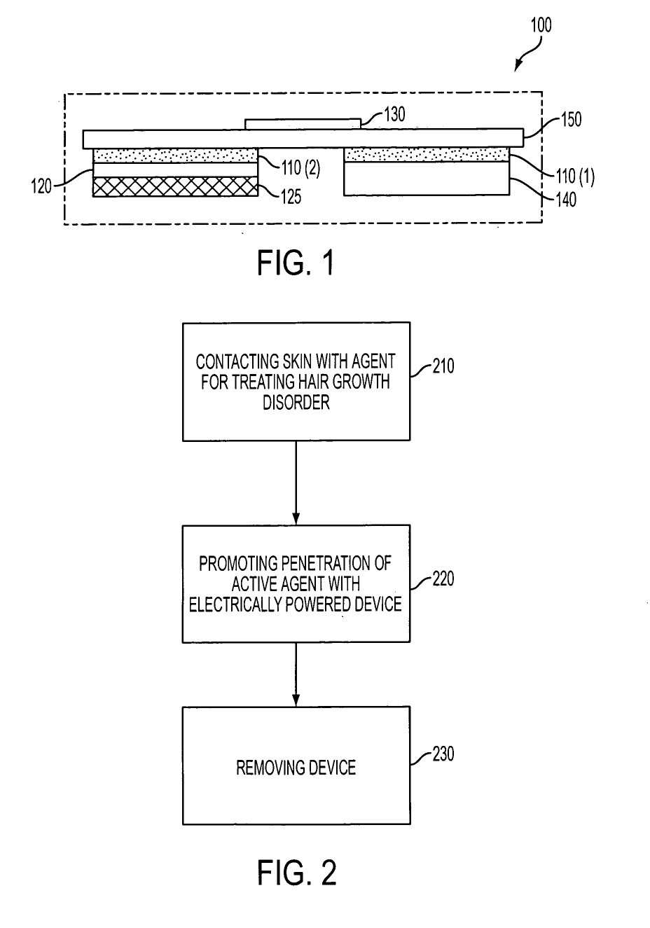

FIG. 1 shows a schematic view of a fully integrated electrically powered device, wherein the electrically powered device is an iontophoretic patch device for treatment of a hair growth disorder according to one embodiment of the present invention. In this embodiment, patch 100 may comprise first electrode 110(1), identified as “cathode,” second electrode 110(2), identified as “anode,” and electrochemical cell 130 as the power supply of patch 100. Optionally, patch 100 may include a plurality of cathodes 110 (1), a plurality of anodes 110 (2) and a plurality of power supplies 130. Patch 100 may also comprise conductive layer/s 120 and 140 to provide an interfacing layer between patch 100 and a body area of a subject. One of or both conductive layer/s 120 and 140 optionally include active agents for hair growth disorder 125. As shown in FIG. 1, electrodes 110(1) 110(2), conductive layers 120, 140, active agents for hair growth disorder 125, and electrochemical cell may be supported on substrate 150. Electrode 110(1) may be disposed in any suitable way on substrate 150 in spaced relation to electrochemical cell 130 and electrode 110(2) to define a gap between the two electrodes. Conductive layer 120 including active agents for hair growth disorder 125 may optionally be disposed on electrode 110(2) or 110(1) or on both electrodes 110(1) and 110(2). In an alternative embodiment, patch 100 does not include conductive layer 120 (or conductive layer 140). In this alternative embodiment active agents for hair growth disorder 125 can optionally be accommodated in a holding component, such as a retainer, chamber, sponge, separator, non-woven material or substrate base layer (not shown in figure), which is attached to electrode 110(1) or electrode 110(2). Preferably, holding component for holding and storing the formulation has a pore size and a pore density, and contacts a first region of the body area and the active electrode. Alternatively, active agents for hair growth disorder can be accommodated on electrode 110(1) or 110(2) in any other suitable way.

Preferably the device 100, including first electrode 110(1), second electrode 110(2) and electrochemical cell 130, is thin, and flexible, to suit the contour of a skin portion of a subject.

As noted, the embodiment depicted in FIG. 1 is a fully integrated patch device. An embodiment of the patch used as part of a kit in accordance with the present invention is similar to the embodiment of the fully integrated patch device depicted in FIG. 1. However, in this alternative embodiment, the conductive layer 120 of the patch optionally (when it is part of a kit) does not include an active agent for treatment of a hair growth disorder 125. Alternatively, active agent for treatment of a hair growth disorder 125 accommodated in conductive layer 120 is disposed in a separate holding component, which is not integrally attached to the patch. Optionally, separate holding component can be attached to patch just before use, such as for example when separate holding component is a chamber. Alternatively, separate holding component can be applied onto body area, such as for example when separate holding component is a sponge or other type of material absorbing device. Alternatively, active agent for treatment of a hair growth disorder 125 accommodated in conductive layer 120 is applied directly onto body area or onto electrode, without use of a separate holding component.

Any power supply 130, which provides an electrical potential of between about 0.2 Volt and about 50 Volt can be used according to the present invention. Yet, in a preferred embodiment, power supply 130 is an electrical battery, providing an electrical potential of between about 0.5 Volt and 20 Volt. Most preferably, power source provides electrical potential (voltage) in the range between about 0.5V and about 9V. Such electrical potential can be supplied by a single electrochemical cell or a number of electrochemical cells, linked together, to afford the desirable voltage. In a further preferred embodiment, the electrical potential is adjusted, to satisfy at least two of the following criteria:

The voltage is adjusted to enable an iontophoretic delivery of the active agent into the skin. For that purpose, voltage is adjusted to provide an electrical current of between about 0.002 mAmp/cm2 and 10 mAmp/cm2.

The voltage is adjusted to minimize the penetration of the active agent through the skin. Thus, using an in-vitro skin penetration model, and applying the selected voltage, the amount of active agent found in the skin is higher than the respective amount found in the receiving compartment of a modified Franz cell. Thus, in a preferred embodiment, the voltage is in the range between about 0.5V and about 20V; and in a more preferred embodiment, the voltage in the range between about 1.5V and about 6V.

The voltage is adjusted to minimize skin irritation, which may result from excessive electrical current, passing into and through the skin. Thus, in a preferred embodiment, the voltage is in the range between about 0.5V and about 20V; and in a more preferred embodiment, the voltage in the range between about 1.5V and about 6V.

Preferably, power supply 130 is thin and flexible and disposable. Power supply 130 is optionally any suitable size, shape or thickness. Preferably, power supply 130 is at least one electrochemical cell. The term ‘electrochemical cell’ as used herein includes any suitable open, closed or semi-open cell in any suitable physical or chemical state in which chemical energy is converted to electric energy by a spontaneous electron transfer reaction. The term includes cells with non-spontaneous reactions, cells, cells with spontaneous reactions, galvanic cells, electrolytic cells and a combination thereof. According to a preferred embodiment of the present invention, the electrochemical cell of the patch may be a thin, flexible and disposable electrochemical cell. Preferably, power supply thickness should not exceed about 4 mm and more preferably, power supply thickness should be less than about 2 mm and most preferably up to about 1 mm. In a further preferred embodiment, power supply 130 is at least one electrochemical cell, the electrochemical cell including a first layer of insoluble negative pole, a second layer of insoluble positive pole and a third layer of aqueous electrolyte, the third layer being disposed between the first and second layers and including: (a) a deliquescent material for keeping the open cell wet at all times; (b) an electroactive soluble material for obtaining required ionic conductivity; and (c) a water soluble polymer for obtaining a required viscosity for adhering the first and second layers to the third layer. Such a power source is described in U.S. Pat. Nos. 5,652,043, 5,811,204 and 5,897,522, which are incorporated herein in their entirety. Briefly, the electrochemical cell described in the above-identified U.S. Patents is preferably an open liquid state, electrochemical cell, which can be used as a primary or rechargeable power source for various miniaturized and portable electrically powered devices of compact design. However, the use of any power source consistent with a flexible wearable device is within the scope of the invention.

Optionally, power supply 130 in patch 100 is a single electrochemical cell. However, power supply 100 need not be limited to one cell, but may include a plurality of connected electrochemical cells, a plurality of batteries, and/or electronics configured to increase, control, and change phase of the supplied electric current and wherein the power supply is thin and flexible. Electrochemical cell 130 in patch 100 preferably provides electrical potential (voltage) to the desired body area of the subject.

Yet, in another preferred embodiment, the thin and flexible electrochemical cell consists of plurality of self-contained, serially connected galvanic power sources, as described for example in U.S. Pat. No. 6,421,561.

Cathode and anode electrodes 110(1) and 110(2) are preferably composed of a conductive material. Any suitable conductive material, including metallic and non-metallic materials, may optionally be used as electrode materials, such as, but not limited to silver, silver/silver chloride, graphite, zinc, copper, carbon, platinum, manganese dioxide or a combination thereof. In a preferred embodiment, at least one of the electrodes may comprise silver, zinc and silver/silver chloride and carbon. Any other conductive element or compound, including metal and non-metal materials, can be used as electrode materials. The electrodes may optionally be provided in any suitable form, such as, but not limited to as thin sheets, linked to the power source, or printed onto a base member substrate in spaced relation to each other to define a gap therebetween. The electrode area can be continuous, or formed in any shape. Preferably, electrodes will be formed in a configuration suitable for diffuse area treatment, such as, but not limited to in a concentric configuration or spiral configuration. Diffuse area treatment is preferable, as the body area affected by hair loss or hair growth may be large. Optionally, patch can include a plurality of anodes and a plurality of cathodes. Such a multi-electrode patch facilitates providing simultaneously a plurality of treatments with one active agent in different body areas or the same body area.

In the embodiment shown in FIG. 1, anode electrode 110(2) is active. However, either anode electrode 110(2), cathode electrode 110(1), or both electrodes may be active for delivering an active agent for treatment of a hair disorder. Thus, at least the features of the patch of the present invention described above are the same regardless of whether the patch is a fully integrated patch device, or a patch device included as part of a kit.

The interfacing layer between the device, and the skin is preferably a conductive layer. Without derogating from the generality of optional interfacing materials, conductive layers 120 and 140 may optionally be any suitable conductive composition, such as an aqueous gel, hydrogel or a conductive adhesive.

Substrate base layer 150 is optionally any suitable material, which can accommodate the patch components. Suitable materials include, but are not limited to woven material, non-woven material, polymers, conducting material, non-conducting material, paper, cardboard, plastic, synthetic materials, natural materials, fabric, metals, wood, glass, Perspex, or a combination thereof. Preferably, substrate material is a non-conductive material. More preferably, substrate is made from polyester. Optionally, substrate base layer 150 can be made up of a plurality of substrate base layers 150, which can be stacked or connected in a co-planar way by any suitable attachment means. Preferably, substrate layer 150 is made up of one continuous piece of substrate layer 150. Optionally, substrate base layer 150 can be any suitable size, shape or color.

Optionally, substrate base layer 150 includes an attachment means, which readily facilitates attaching device 100 to the desired body area. Attachment means include but are not limited to any suitable attachment means for example, conductive adhesive, adhesive strip, suction means and combinations thereof. In the embodiment of FIG. 1, patch 100 is configured to attach to the body area by conductive layer 140. In alternate embodiments, the patch may be attached to the body area by other attachment means such as, but not limited to the frame of the substrate and/or attachment means on the substrate.

The terms “iontophoretic patch” and “iontophoretic device”, as used herein interchangeably, stand for an electrically powered device, enhancing the dermal delivery and/or effectiveness of agents by mechanisms such as iontophoresis, electroosmosis, electrotransport, electroporation and the like. This term will further stand for any electrically powered device that provides direct current or alternating current electrical stimulation.

Hair growth agents, according to the present invention are such agents that influence hair growth, either by enhancing growth or inhibiting growth. Generally, hair growth active agents according to the present invention include any known active agents that influence hair growth, as exemplified in the background of this application. Such agents need to be bioavailable at their target site of action, i.e., the skin and pilosebaceous unit, in therapeutically active concentrations and thus, their application under iontophoresis can result in enhanced efficacy.

The following are specific examples of classes of agents, which can be used as hair growth agents herein.

“Androgen inhibitors” is one class of such compounds, which may be effective in the treatment of androgen-related hair growth disorder, including hair loss (e.g., androgenic alopecia) and androgen-related excessive body and face hair growth, such as in the case of hirsutism and hypertrichosis.

This class includes substances of any androgen hormone, which is involved in the control of hair growth of the body and face. Such hormones include Testosterone and DHEAS, which are biologically active. Testosterone is converted to a more potent androgen, namely dihydrotestosterone (DHT). Androstenedione and DHEA are not androgenic, however, upon their conversion to testosterone, they become androgenically effective and thus, in the context of the present invention, they are included in the general definition of “androgens”. Without derogating from the generality of the term, “androgen inhibitors” include agents which exert their effect by a mechanism such as (1) inhibition of the synthesis of such hormones; (2) binding to such hormones, rendering them inactive; (3) inhibiting their presentation to the respective hormone receptors; (4) androgen receptor binding; as well as other mechanisms, which lead to the inhibition of the inductive effect of androgen hormones on body and face hair growth. One preferred class of androgen inhibitors includes inhibitors of the enzyme 5-alpha-reductase, including, by way of example, steroid hormones (e.g., finasteride and spironolactone), dicarboxylic acids, having 7-13 carbon atoms in their carbon backbone (e.g., azelaic acid), and derivatives thereof; imidazoles, such as ketoconazole and phytosterols, such as found in herbal extracts (e.g., saw palmetto extract).

Androgen inhibitors, delivered using a patch or kit according to the present invention, are also useful in the treatment of other disorders of the pilosebaceous gland, such as acne.

Hair growth stimulants, according to the present invention, include agents that induce the growth of hair. Such agents may be effective by stimulating blood flow; or adding nutrients, such as vitamins, minerals, amino acids, nucleotides and nucleosides that hair needs to grow properly.

There is a variety of hair growth inhibitor compounds that can be effective in inhibiting hair growth, provided that they are bioavailable in the skin, in therapeutic levels.

Other agents useful in the control of hair growth include inhibitors of enzymes, such as inhibitors of ornithine decarboxylase, S-adenosylmethionine decarboxylase, gamma-glutamyl transpeptidase, and transglutaminase, are detailed, for example, in Breuer et al., U.S. Pat. No. 4,885,289; Shander, U.S. Pat. No. 4,720,489; Ahluwalia, U.S. Pat. No. 5,095,007; Ahluwalia et al., U.S. Pat. No. 5,096,911; Shander et al., U.S. Pat. No. 5,132,293; and Shander et al., U.S. Pat. No. 5,143,925.

Yet, another class of hair growth inhibitors are such agents, which inhibit directly or indirectly the growth of keratin tissue. Aminoacyl-tRNA syntheses are examples of such agents, being a family of enzymes that are involved in cellular protein synthesis. In particular, the enzymes participate in the activation of amino acids and the subsequent linkage of the amino acids to corresponding tRNAs. It has been found that unwanted growth can be reduced by applying to the skin a dermatogically acceptable composition including an inhibitor of an aminoacyl-tRNA synthetase in an amount effective to reduce hair growth, as detailed, for example, in U.S. Pat. No. 5,939,458.

Growth factors are proteins that bind to receptors on the cell surface, with the primary result of activating cellular proliferation and/or differentiation. Cytokines are signaling molecules that cells use to communicate with each other. They are involved in the control and modulation of tissue differenciation and growth, including hair growth. By way of example, growth factors and cytokines, which affect hair growth disorders include epidermal growth factor (EGF), Fibroblast Growth Factors (FGF), tumor necrosis alpha, interferon, various interleukines, vascular endothelial growth factor (VEGF), nitric oxide, insulin-like growth factors (e.g., IGF-I and IGF-II) and parathyroid hormone related peptide (PTHrP).

As modulators of tissue proliferation and/or differentiation and/or growth, growth factors can affect hair growth, thereby modulating the hair growth cycle, rate of growth and loss of hairs. Thus, growth factors and cytokines are considered “hair growth agents” according to the present invention. By including such agents in the kit of the present invention, their effect may be enhanced by the device of the present invention, to attain treatment of a hair growth disorder, including excessive hair growth and alopecia. Hair removal agents can also be used with the device and/or kit of the present invention to enhance hair removal.

While the most common baldness is male pattern baldness, there are additional types of baldness, which can be treated using a device and/or kit of the present invention comprising a hair growth agent and an iontophoretic device. In alopecia greata, the affected hair follicles are mistakenly attacked by a person's own immune system, resulting in the arrest of the hair growth stage. Thus, a kit comprising an agent that affects the immune system, via different mechanisms, thereby influencing this hair growth disorder, and an iontophoretic device, is suitable for treatment of alopecia greata, alopecia totalis and alopecia universalis.

Similar treatments, using all types of hair growth agent, together with an iontophoretic device, can be suitable for the treatment of other types of baldness, such as aplopecia universalis and alopecia totalis. Examples of possible hair growth agents for such hair growth disorders are corticosteroids, anthralin, minoxidil, retinoids, such as tretinoin, immunomodulators such as cyclosporin, tacrolimus and pimecrolimus and triamcinolone acetonide.

Optionally, active agents for the treatment of hair growth disorders according to the present invention may be part of a formulation, placed in the interface area between one or both of the electrodes of the device. Providing that they possess a certain degree of water solubility, the active agents can be mobilized from the formulation towards the body surface, via the electromotive forces of iontophoresis and/or electro-osmosis. The term ‘formulation’ as used herein includes any type of suitable formulation, which can accommodate an active agent for the treatment of a hair growth disorder. The term includes conductive layers, such as aqueous gel or hydrogel. The term further includes any pharmaceutical or cosmetic active or inactive formulation, including active ingredients, solvents, fragrance and additives. It would be apparent to those of ordinary skill in the art of cosmetics and dermatology that additives to such formulations may be selected from but are not limited to the group consisting of water, surfactants, emulsifiers, diglycerides, triglycerides, stabilizing agents, thickening agents, alpha-hydroxy carboxylic acids, antioxidants, preservatives, moisturizers, petroleum, mineral oil, glycerol, ethanol, propanol, isopropanol, butanol, polymeric gelling agents, flavoring, colorant and odorant agents and other formulation components, used in the art of pharmaceutical and cosmetic formulary.

The therapeutic system of the present invention may further comprise any additional therapeutic agents or active agents or additives, which may contribute to the therapy of the disorder or another related or unrelated disorder.

In an embodiment, wherein the active agent for the treatment of a hair growth disorder is placed in the interface area between one or both of the electrodes, the formulation containing the active agent for the treatment of a hair growth disorder can optionally be applied directly onto the skin between the two electrodes, or alternatively the active agent is disposed in a holding component, such as, but not limited to a sponge placed between the two electrodes or applied onto the substrate between the two electrodes. Preferably, the formulation is contained in a conductive layer, such as but not limited to a hydrogel. In effect there are two current pathways in this embodiment, one current pathway between anode and cathode and a second current pathway between the active electrode/s and skin. Current flowing from the anode to the cathode readily facilitates surface treatment with the active agent onto the skin. In addition, current flowing from the active electrode/s to the skin readily facilitates iontophoretic delivery and/or electroosmosis of active agent from the formulation into the skin. Therefore, this embodiment readily facilitates a combination of both surface treatment with the active agent and iontophoretic delivery and/or electroosmosis delivery into the skin of the active agent for transdermal and dermal treatment.

Optionally, the active agent or formulation containing the active agents can be applied directly onto the skin or can be applied onto the electrode or to the interface area between one or both of the electrodes.

Yet, in another preferred embodiment, hair growth is stimulated by means of an electrically powered device, that provides direct current or alternating current electrical stimulation, with or without an active agent. Without wishing to be limited by a single mechanism, electric current can modulate and accelerate hair growth by effecting the neural activations in the epidermis. The neural sensors, such as nociceptors type C secrete substance P, which is known to accelerate hair growth. In addition, an electrically powered device, in combination with a formulation, such as, but not limited to hydrogel comprising potassium can accelerate this secretion. The potassium can influence directly the hair follicle, facilitating acceleration of hair growth. Therefore, electric current from an electrically powered device alone or in combination with potassium can induce hair follicle activity and proliferation.

The treatment according to the present inventions may be beneficial in all body areas. Preferably, the body area to be treated with the device or kit and active agent of the present invention includes, but is not limited to any bodily area, such as skin where there is hair or hair follicles or where hair is desired and/or sebaceous glands. Preferably, the device and kit of the present invention is for use on humans. Optionally, the device and kit of the present invention can be used on non-humans.

In a preferred embodiment, wherein the patch is thin, flexible and versatile in shape and form, the preferred devices of the present invention can be designed to fit any area of the body surface and to have any desirable size, according to the area having the disorder.

The patch of the present invention may be made using any suitable techniques. Preferably, the patch of the present invention is a printed patch, wherein the electrodes and power supply are printed onto the substrate using any suitable printing technology.

FIG. 2 is a flowchart of a method according to embodiments of the present invention. The flowchart applies to a method using a fully integrated patch, or to a method using a kit including a patch. First, a subject may contact (210) a body area with an active agent for treatment of a hair growth disorder (which may or may not be part of a patch). The subject may promote (220) penetration and/or generation of active agent for treatment of a hair growth disorder onto and/or into the body area through the use of an electrically powered patch. The patch is removed from the body area (230) at the end of the treatment time. Optionally end of treatment time is determined by depletion of the active agent and/or sufficient therapeutic effect of the treatment or by a predetermined time. The treatment can optionally be a one-time treatment or can be repeated in suitable time intervals any suitable number of times.

Therefore, according to one embodiment, the kit can be used sequentially, whereby the hair growth active agent is first applied, followed by application of the iontophoretic patch. Yet, according to another embodiment of the same invention, the hair growth agent is located on the device, which is applied simultaneously with application on the device on the target area of the skin.

While the principles of the invention have been discussed in relation to exemplary embodiments discussed herein, it is understood that the principles of the invention are not limited thereto.

### EXAMPLE 1

### Treatment of Hair Loss with an Androgen Inhibitor

Preferably, the device or kit is used with a formulation including an androgen inhibitor resulting in treatment of hair loss. Optionally, any suitable formulation including an androgen inhibitor is envisioned for use with the patch device. In some circumstances it may be preferable to have a mixture of the androgen inhibitor and an additional active ingredient or additive, such as but not limited to any suitable hair growth stimulant agent.

The androgen inhibitor is preferably contained in the hydrogel to form a mixture. The mixture can optionally be applied directly to the desired area of the body. Alternatively, the mixture is preapplied, such as, but not limited to, during manufacture of the patch, onto the electrode/s of the patch and therefore the patch is ready for use. Further, the aqueous hydrogel can be contained in a separator, which is integrally formed with the patch or in a holding component disposed between the two electrodes. Still further, the mixture can be contained in a separate, non-integral holding component, such as a retainer. The retainer is then optionally connected to the patch before use.

A typical method of treatment includes the following steps of adhering the patch of the present invention to the desired area of the body, such as, but not limited to the head. One area or more than one area of the body can be treated at the same time using a plurality of patches or the same patch with a plurality of electrodes. If the kit of the present invention, which includes a separate retainer is being used, the retainer which preferably contains the mixture is first attached to the patch before adhering to the skin. In the case where a patch of the present invention is being used, which does not include hydrogel, the aqueous hydrogel can be applied directly to the place of treatment on the body of the subject. In the case that the hydrogel is contained in the patch, the patch is ready to use.

Adhering the patch to the skin closes the electrical circuit. Current will then flow, resulting in iontophoresis and electrical stimulation of the skin and dermal and transdermal penetration of the androgen inhibitor. The duration of treatment is determined according to severity of condition and other skin factors. Treatment is terminated by removal of the patch. Alternatively, treatment is terminated by depletion of the battery, at least one of the electrodes or depletion of the androgen inhibitor.

Typically, the treatment is repeated in selected time intervals. At the beginning, the treatment is usually repeated more frequently, such as a few times a week and then for maintenance of the resulting hair growth effect, the treatment is repeated less frequently.

### EXAMPLE 2

### Treatment of Hair Loss with an Electrically Powered Device

An iontophoresis patch with a hydrogel comprising potassium is used. A typical method of treatment includes the following steps of adhering the patch of the present invention to the desired area of the body, such as, but not limited to the head. One area or more than one area of the body can be treated at the same time using a plurality of patches or the same patch with a plurality of electrodes. If the kit of the present invention, which includes a separate retainer is being used, the retainer which preferably contains the hydrogel is first attached to the patch before adhering to the skin. In the case where a patch of the present invention is being used, which does not include hydrogel, the aqueous hydrogel can be applied directly to the place of treatment on the body of the subject. In the case that the hydrogel is contained in the patch, the patch is ready to use.

Adhering the patch to the skin closes the electrical circuit. Current will then flow, resulting in electrical stimulation of the skin and secretion of substance P. The duration of treatment is determined according to severity of condition and other skin/hair factors. Treatment is terminated by removal of the patch. Alternatively, treatment is terminated by depletion of the battery or at least one of the electrodes.

Typically, the treatment is repeated in selected time intervals. At the beginning, the treatment is usually repeated more frequently, such as a few times a week and then for maintenance of the resulting hair growth effect, the treatment is repeated less frequently.

It will be appreciated by persons skilled in the art that the present invention is not limited to what has been particularly shown and described hereinabove. Rather, the scope of the present invention is defined by the appended claims and includes both combinations and subcombinations of the various features described hereinabove as well as variations and modifications thereof which would occur to persons skilled in the art upon reading the foregoing description. Accordingly, it is intended to embrace all such alternatives, modifications and variations that fall within the spirit and broad scope of the appended claims.

1. A kit for treating a hair growth disorder, the kit comprising:
(a) at least one formulation comprising a hair growth modulating agent; and
(b) at least one electrically powered device for promoting delivery of the hair growth modulating agent into and/or onto the body area.

2. The kit of claim 1, wherein the device is an iontophoretic patch.

3. The kit of claim 2, wherein the patch comprises:
(a) at least one first electrode in electrical contact with a first region of the body area;
(b) at least one second electrode in electrical contact with a second region of the body area; and
(c) at least one power supply for supplying electrical energy to the first electrode and the second electrode, wherein the power supply is supported on a base member.

4. The kit of claim 3, wherein the power supply comprises at least one electrochemical cell.

5. The kit of claim 4, wherein the electrochemical cell is thin and flexible.

6. The kit of claim 5, wherein the electrochemical cell is up to 1 mm thick.

7. The kit of claim 3, wherein the first electrode is an anode and the second electrode is a cathode.

8. The kit of claim 4, wherein the electrochemical cell comprises:
(a) a first layer of insoluble negative pole;
(b) a second layer of insoluble positive pole; and
(c) a third layer of aqueous electrolyte disposed between the first and second layers and including:
(i) a deliquescent material for keeping the electrochemical cell wet at all times;
(ii) an electroactive soluble material for obtaining ionic conductivity; and
(iii) a water-soluble polymer for obtaining a desired viscosity for adhering the first and second layers to the third layer.

9. The kit of claim 4, wherein the electrochemical cell has open architecture.

10. The kit of claim 7, wherein the patch comprises an active electrode, and the active electrode is the anode, the cathode or both the anode and the cathode.

11. The kit of claim 1, wherein the formulation is disposed on the active electrode.

12. The kit of claim 1 further comprising a holding component for holding and storing the formulation and having a pore size and a pore density, the holding component contacting a first region of the body area and the active electrode.

13. The kit of claim 12, wherein the holding component is selected from the group consisting of a sponge, separator, non-woven material or substrate base layer.

14. The kit of claim 1, wherein the hair growth modulating agent is applied directly to the body area.

15. The kit of claim 1, wherein the hair growth modulating agent is disposed in the interface area between the electrodes.

16. The kit of claim 15, wherein the kit promotes surface treatment, dermal treatment and transdermal treatment of the body area with the hair growth modulating agent.

17. The kit of claim 1, further comprising:
a conductive layer comprising a conductive composition disposed between the patch and the body area, for providing a conductive interfacing layer between the patch and the body area.

18. The kit of claim 17, wherein the conductive composition is a conductive adhesive.

19. The kit of claim 17, wherein the hair growth modulating agent is contained in the conductive composition.

20. The kit of claim 17, wherein the patch comprises an active electrode and a counter electrode, and the conductive layer is disposed on the active electrode.

21. The kit of claim 1, wherein the treatment is selected from the group consisting of curing, alleviating condition, alleviating symptoms, prevention and a combination thereof.

22. The kit of claim 2, wherein the patch is thin and flexible.

23. The kit of claim 2, wherein the patch is made by a printing method.

24. The kit of claim 2, where the patch further comprises attachment means.

25. The kit of claim 1, wherein the hair growth disorder is selected from the group consisting of any type of hair loss and excessive hair growth, acne, male pattern baldness, androgenic alopecia, alopecia greata, alopecia totalis and alopecia universalis and a combination thereof.

26. The kit of claim 1, wherein the hair growth modulating agent is selected from the group consisting of a hair stimulating agent, a hair growth inhibitory agent, a hair removal agent and a combination thereof.

27. The kit of claim 1, wherein the hair growth modulating agent is selected from the group consisting of androgen inhibitors, inhibitors of ornithine decarboxylase, S-adenosylmethionine decarboxylase, gamma-glutamyl transpeptidase, and transglutaminase, keratin inhibitors, hair growth stimulants, vitamins, growth factors, cytokines, potassium formulations, corticosteroids, minerals, immunomodulators and any combination thereof.

28. The kit of claim 1, wherein the hair growth modulating agent is an agent, which acts directly on the hair follicle.

29. A kit for treating a disorder of the pilosebaceous unit, comprising:
(a) at least one formulation comprising an androgen inhibitor; and
(b) at least one electrically powered device for promoting delivery of the androgen inhibitor into and/or onto a body area comprising pilosebaceous units.

30. The kit of claim 29, wherein the device is an iontophoretic patch.

31. The kit of claim 30, wherein the patch comprises:
(a) at least one first electrode in electrical contact with a first region of the body area;
(b) at least one second electrode in electrical contact with a second region of the body area; and
(c) at least one power supply for supplying electrical energy to the first electrode and the second electrode, wherein the power supply is supported on a base member.

32. The kit of claim 31, wherein the power supply comprises at least one electrochemical cell.

33. The kit of claim 32, wherein the electrochemical cell comprises:
(a) a first layer of insoluble negative pole;
(b) a second layer of insoluble positive pole; and
(c) a third layer of aqueous electrolyte disposed between the first and second layers and including:
(i) a deliquescent material for keeping the electrochemical cell wet at all times;
(ii) an electroactive soluble material for obtaining ionic conductivity; and
(iii) a water-soluble polymer for obtaining a desired viscosity for adhering the first and second layers to the third layer.

34. A device for treatment of a disorder of the pilosebaceous unit comprising a flexible, wearable patch conformable to the contour of a body area surface and comprising:
(a) at least one first electrode adapted to communicate an active substance into the body area by application of an electrical current on the body area surface;
(b) at least one second electrode, facilitating closing of electrical circuit with the body area surface;
(c) at least one power source for providing a current and voltage, connected through a conductive media to the first and second electrode; and
(d) a hair growth modulating agent and or an anti-acne agent for treatment of the disorder.

35. The device of claim 34, further comprising a holding component for holding the hair growth modulating agent and/or anti acne agent.

36. The device of claim 34, wherein the hair growth modulating agent and/or anti acne agent is applied directly to the body area.

37. The device of claim 34, wherein the hair growth modulating agent and/or anti acne agent is disposed in the interface area between the at least one first electrode and the at least one second electrode.

38. A device for treatment of a disorder of the pilosebaceous unit comprising a flexible, wearable patch conformable to the contour of a body area surface and comprising:
(a) at least one first electrode adapted to communicate an active substance into the body area by application of an electrical current on the body area surface;
(b) at least one second electrode, facilitating closing of electrical circuit with the body area surface; and
(c) at least one power source for providing a current and voltage, connected through a conductive media to the first and second electrode;
wherein the electrical current stimulates hair growth.

39. The device of claim 38 further comprising a formulation comprising potassium.

40. The device of claim 38, further comprising a substrate base layer.

41. A method for treating a hair disorder of a body area with an agent for modulating hair growth comprising:
(a) providing at least one electrically powered device for promoting delivery of the hair growth modulating agent into and/or onto the body area;
(b) contacting the body area with the device for a time period wherein the device promotes hair growth modulating agent penetration of the body area surface and underlying tissues;
(c) penetrating of the hair growth modulating agent into and/or onto the body area to treat the hair disorder; and
(d) removing the device from the body area.

42. The method of claim 41 wherein a formulation comprising the hair growth modulating agent is applied onto affected body area prior to the step of contacting the body area with the device.

43. The method of claim 41 wherein a formulation comprising the hair growth modulating agent is applied onto affected body area simultaneously with the device.

44. The method of claim 43, wherein the formulation is disposed on the device.

45. The method of claim 41, wherein the hair growth disorder is baldness.

46. The method of claim 41, wherein the hair growth disorder is selected from the group consisting of male pattern baldness, androgenic alpecia, alopecia greata, alopecia totalis and alopecia universalis and a combination thereof.

47. The method of claim 41, wherein the hair growth disorder is excessive hair growth.

48. The method of claim 41, wherein the hair growth disorder is acne.

49. The method of claim 41, wherein the device comprises:
(a) at least one first electrode in electrical contact with a first region of the body area;
(b) at least one second electrode in electrical contact with a second region of the body area; and
(c) at least one power supply for supplying electrical energy to the first electrode and the second electrode, wherein the power supply is supported on a base member.

50. The method of claim 49, wherein the power supply comprises at least one electrochemical cell.

51. The method of claim 50, wherein the electrochemical cell is thin and flexible.

52. The method of claim 51, wherein the electrochemical cell comprises:
(a) a first layer of insoluble negative pole;
(b) a second layer of insoluble positive pole; and
(c) a third layer of aqueous electrolyte disposed between the first and second layers and including:
(i) a deliquescent material for keeping the electrochemical cell wet at all times;
(ii) an electroactive soluble material for obtaining ionic conductivity; and
(iii) a water-soluble polymer for obtaining a desired viscosity for adhering the first and second layers to the third layer.

53. A method for treating a disorder of the pilosebaceous unit comprising:
(a) providing at least one electrically powered device;
(b) contacting the pilosebaceous unit body area with the device for a time period wherein the device provides electrical stimulation to the body area;
(c) stimulating the pilosebaceous unit body area to treat the disorder; and
(d) removing the device from the body area.

54. The method of claim 53, wherein the disorder is a hair growth disorder.

55. The method of claim 53, wherein the disorder is acne.

56. The method of claim 53, further comprising the step of applying a formulation comprising potassium to modulate hair growth.

57. The method of claim 53, wherein the hair growth disorder is baldness.

58. The method of claim 53, wherein the hair growth disorder is selected from the group consisting of male pattern baldness, androgenic alpecia, alopecia greata, alopecia totalis and alopecia universalis and a combination thereof.

59. The method of claim 50, wherein the hair growth disorder is excessive hair growth.

60. The method of claim 53, wherein the device comprises:
(a) at least one first electrode in electrical contact with a first region of the body area;
(b) at least one second electrode in electrical contact with a second region of the body area; and
(c) at least one power supply for supplying electrical energy to the first electrode and the second electrode, wherein the power supply is supported on a base member.

61. The method of claim 60, wherein the power supply comprises at least one electrochemical cell.

62. The method of claim 61, wherein the electrochemical cell is thin and flexible.

63. The method of claim 62, wherein the electrochemical cell comprises:
(a) a first layer of insoluble negative pole;
(b) a second layer of insoluble positive pole; and
(c) a third layer of aqueous electrolyte disposed between the first and second layers and including:
(i) a deliquescent material for keeping the electrochemical cell wet at all times;
(ii) an electroactive soluble material for obtaining ionic conductivity; and
(iii) a water-soluble polymer for obtaining a desired viscosity for adhering the first and second layers to the third layer.
